(19) United States
(12) Patent Application Publication
(10) Pub. No.: US 2005/0117754 A1
(43) Pub. Date: Jun. 2, 2005

(54) Active noise cancellation helmet, motor vehicle system including the active noise cancellation helmet, and method of canceling noise in helmet

(76) Inventor: Atsushi Sakawaki, Shizuoka (JP)

Correspondence Address: KEATING & BENNETT LLP, Suite 312, 10400 Eaton Place, Fairfax, VA, 22030, US

(21) Appl. No.: 10/998,224

(22) Filed: Nov. 26, 2004

(30) Foreign Application Priority Data
Dec. 2, 2003 (JP) 2003-403745

(51) Int. Cl. A61F 11/06; G10K 11/16; H03B 29/00

(52) U.S. Cl. 381/71.6; 381/72

(57) ABSTRACT

An active noise cancellation helmet includes a detection unit which detects noise in a helmet body, and a sound outputting unit which outputs a sound for canceling the noise detected by the detection unit. A control signal is generated by processing an output signal of the detection unit through computation. The control signal is amplified by an amplification unit, and applied to the sound outputting unit. A ratio of sound pressures in different frequency ranges is determined on the basis of the output signal of the detection unit. A gain of the amplification unit is adjusted on the basis of the sound pressure ratio so as to approximate a spectrum of the output signal of the detection unit to a predetermined target spectrum.

### BACKGROUND OF THE INVENTION

1. Field of the Invention

The present invention relates to an active noise cancellation helmet, a motor vehicle system including the active noise cancellation helmet, and a method of canceling noise in the helmet.

2. Description of the Related Art

In recent years, attention has been directed to an active noise cancellation or active noise control (ANC) technique for canceling noise by secondarily generating a sound wave having the same amplitude as a noise sound wave in an inverted phase and causing interference between the secondary sound wave and the noise sound wave. With recent advancement of a digital signal processing technique, the ANC technique has found applications in a variety of fields.

One exemplary application of the active noise cancellation technique is a headset with an active noise cancellation capability as disclosed in WO95/00946.

The headset is a feedback type active noise cancellation device, which includes microphones respectively provided inside and outside of a sound field, i.e., inside and outside of the ear cups of the headset. In order to improve the noise cancellation capability, the device uses band-pass filters having the same frequency characteristics to compare the sound pressures of noises observed in a specific frequency band (e.g., a resonance frequency band) inside and outside of the sound field with each other and adjust a control gain (amplifier gain) so as to keep the sound pressure ratio of the noises at a constant level.

Since the active noise cancellation technique described in WO95/00946 is directed to the headset, it is difficult to apply the active noise cancellation technique to a helmet which is used in a significantly different sound field from that of the headset.

In the case of the headset, a source of noise to be canceled is located far from the headset. In the case of the helmet, multiple noise sources are present within the helmet. That is, the noise to be canceled in the helmet is mainly a wind noise to which a rider on a two-wheeled motor vehicle (e.g., a motor cycle) is subjected during traveling. Noises generated by the vehicle and road noise also enter into and are present in the helmet. Therefore, it is impossible to provide a sufficient noise cancellation effect simply by comparing the noises observed inside and outside of the sound field in the case of the helmet in which the multiple noise sources are present and generate a complicated sound field.

Further, the noise cancellation effect provided by the helmet varies from user (helmet wearer) to user due to individual differences in the shape of a user's face and head, and the like. More specifically, the space formed between the helmet and a user's head depends on the shapes of the user's face and head, thereby causing individual differences in the noise cancellation effect. Firstly, it is known that sound conduction characteristics (gain characteristics) observed in a user's ear space differ among individuals (see FIG. 13). This individual difference corresponds to a difference in a sound conduction system, i.e., a difference in a frequency conduction function to be controlled (hereinafter referred to as “auditory sound conduction function”). Secondly, it is also known that the inclination of a wind noise spectrum differs among individuals (see FIG. 14). That is, the wind noise spectrum is typically such that a sound pressure is reduced as a frequency increases, and differs among individuals.

FIG. 13 is a graph showing the individual difference in the auditory sound conduction function. The graph shows the results of an experiment by way of example. As shown in FIG. 13, frequency spectra (relationships between a gain and a frequency) for different users have substantially the same profile, but are different in the gain of the conduction function. In FIG. 13, a difference in the gain between a user Q₁ and a user Q₂ is about 9 dB at the maximum (the gain differs by a factor of approximately three). If the gain differs by a factor of three, the amplitude of an output signal of the microphone differs by a factor of three even with a sound of the same amplitude being output from a speaker.

Where the gain to be controlled differs among individuals, the control gain should be correspondingly adjusted. If the control gain is adjusted evenly without consideration of the individual difference, the control gain is excessively effective thereby resulting in divergence depending on the user, or conversely, the control gain is excessively ineffective thereby reducing the noise cancellation effect to a level that is lower than expected without divergence. For example, the control gain K for the user Q₁ is three times as effective as the control gain K for the user Q₂. Therefore, if control gain adjustment adapted for the user Q₂ is carried out for the user Q₁, the control gain is excessively effective thereby resulting in divergence. On the other hand, if control gain adjustment adapted for the user Q₁ is carried out for the user Q₂, the effectiveness of the control gain is reduced by a factor of three thereby reducing the noise cancellation effect to a level that is lower than expected without divergence.

FIG. 14 is a diagram showing the individual difference in the inclination of the wind noise spectrum. As shown in FIG. 14, the sound pressure of the wind noise is generally reduced as the frequency increases, and is generally increased as the frequency decreases. However, the inclination of the wind noise spectrum differs among individuals. In FIG. 14, the inclination of the spectrum for the user M₁ is less steep than the inclination of the spectrum for the user M₂. As the inclination decreases, the proportion of a high frequency component in the whole wind noise is increased. Where the inclination of the wind noise spectrum differs among individuals, adaptive control gain adjustment is also required as will be described.

However, the individual differences are not taken into consideration in the active noise cancellation technique described in WO95/000946, making it possible to efficiently perform the active noise cancellation control according to the user. That is, the ratio of the noises observed in the specific frequency band inside and outside of the sound field is merely controlled, so that an individual difference in conduction rate inside and outside of the ear cup cannot be accommodated.

Particularly in the case of the helmet, the individual differences are more liable to occur and, therefore, it is desirable to perform the control to accommodate the individual differences for improvement of the noise cancellation effect.

### SUMMARY OF THE INVENTION

In order to overcome the problems described above, preferred embodiments of the present invention provide an active noise cancellation helmet which provides a sufficient noise cancellation effect irrespective of helmet wearers, a motor vehicle system including the active noise cancellation helmet, and a method of canceling noise in the helmet.

An active noise cancellation helmet according to one preferred embodiment of the present invention includes a detection unit that is arranged to detect noise in a helmet body, a sound outputting unit that is arranged to output a sound for canceling the noise detected by the detection unit, a signal generating unit that is arranged to process an output signal of the detection unit through computation to generate a control signal, an amplification unit that is arranged to amplify the control signal generated by the signal generating unit and to apply the amplified control signal to the sound outputting unit, a sound pressure ratio acquiring unit that is arranged to acquire a ratio of sound pressures in different frequency ranges on the basis of the output signal of the detection unit, and an adjustment unit that is arranged to adjust a gain of the amplification unit on the basis of the sound pressure ratio acquired by the sound pressure ratio acquiring unit so as to approximate a spectrum of the output signal of the detection unit to a predetermined target spectrum. The sound pressure as used herein means an average of amplitudes of sound waves.

With this unique arrangement, the ratio of the sound pressures in the different frequency ranges is acquired on the basis of the output signal of the detection unit (microphone), and the gain of the amplification unit is adjusted on the basis of the acquired sound pressure ratio so that the spectrum of the output signal of the detection unit (microphone) has an optimum profile. Therefore, a control operation can be performed independently of the absolute value of the output signal of the detection unit (microphone) thereby to accommodate an individual difference in auditory sound conduction function. Thus, a sufficient noise cancellation effect can be provided irrespective of helmet wearers (users).

The detection unit is preferably located within the helmet body so as to be located in the vicinity of a user's ear when a user wears the helmet body.

With this unique arrangement, the active noise cancellation is performed based on a sound that is close to a sound actually heard by the user, because the detection unit (microphone) is located in the vicinity of the user's ear. Thus, the accuracy of the active noise cancellation can be improved.

The sound pressure ratio acquiring unit preferably includes a plurality of filters having different frequency characteristics for filtering the output signal of the detection unit, a sound pressure calculating unit that is arranged to process output signals of the respective filters to calculate the sound pressures in the respective frequency ranges, and a sound pressure ratio calculating unit that is arranged to calculate the sound pressure ratio as a control index on the basis of the sound pressures calculated for the respective frequency ranges by the sound pressure calculating unit.

With this arrangement, the sound pressures in the respective frequency ranges are calculated by processing the output signals of the respective filters having different frequency characteristics, and the sound pressure ratio is calculated as the control index on the basis of the sound pressures thus calculated for the respective frequency ranges. Therefore, the sound pressure ratio as the control index can be acquired with a relatively simple circuit.

The sound pressure ratio acquiring unit may include a first acquisition unit that is arranged to acquire a sound pressure in a resonance frequency range on the basis of the output signal of the detection unit, a second acquisition unit that is arranged to acquire a reference sound pressure as a reference for comparison on the basis of the output signal of the detection unit, and sound pressure ratio calculating unit that is arranged to calculate a ratio of the sound pressure acquired for the resonance frequency range by the first acquisition unit to the reference sound pressure acquired by the second acquisition unit for the comparison.

With this unique arrangement, the sound pressure in the resonance frequency range and the reference sound pressure for the comparison are acquired, and the ratio of the sound pressure in the resonance frequency range to the reference sound pressure is calculated. Therefore, the sound pressure ratio as the control index can relatively easily be acquired.

The reference sound pressure to be acquired by the second acquisition unit is preferably a sound pressure in a reference frequency range which is less susceptible to the active noise cancellation than the resonance frequency range and a noise cancellation frequency range in which the noise is canceled by the sound output by the sound outputting unit.

Thus, the sound pressure ratio calculated by the sound pressure ratio calculating unit is dependent upon the sound pressure in the resonance frequency range. Therefore, the level of the sound pressure in the resonance frequency range can be controlled by adjusting the gain of the amplification unit, thereby providing a desired spectrum.

The reference frequency range may be a full frequency range. That is, a sound pressure level in the full frequency range may be used as the reference sound pressure. This is because the sound pressure level in the full frequency range is considered to be rarely dependent on the profile of the spectrum.

The adjustment unit preferably adjusts the gain of the amplification unit so that the sound pressure ratio acquired by the sound pressure ratio acquiring unit is approximated to a target sound pressure ratio corresponding to the predetermined target spectrum. Thus, the spectrum of the output signal of the detection unit is approximated to the target spectrum through simple control, thereby providing a satisfactory noise cancellation effect.

The active noise cancellation helmet preferably further includes an inclination acquiring unit that is arranged to acquire an inclination of the spectrum of the output signal of the detection unit. In this case, the adjustment unit preferably adjusts the gain of the amplification unit on the basis of the sound pressure ratio acquired by the sound pressure ratio acquiring unit and the inclination acquired by the inclination acquiring unit so that the spectrum of the output signal of the detection unit is approximated to the predetermined target spectrum.

With this unique arrangement, the inclination of the spectrum of the output signal of the detection unit (microphone) is further acquired, and the gain of the amplification unit is adjusted on the basis of the sound pressure ratio and the inclination thus acquired. Accordingly, the spectrum of the output signal of the detection unit (microphone) is optimized, making it possible to accommodate an individual difference in the inclination of the spectrum of the output signal of the detection unit (microphone) as well as the individual difference in the auditory sound conduction function. Therefore, a satisfactory noise cancellation effect can be provided irrespective of the physical differences between various helmet wearers.

The adjustment unit preferably includes a target sound pressure ratio setting unit that is arranged to variably set the target sound pressure ratio for the predetermined target spectrum according to the inclination acquired by the inclination acquiring unit, and preferably adjusts the gain so that the sound pressure ratio acquired by the sound pressure ratio acquiring unit is approximated to the target sound pressure ratio set by the target sound pressure ratio setting unit.

With this unique arrangement, the target sound pressure ratio is variably set according to the inclination, and the gain of the amplification unit is adjusted so that the ratio of the sound pressures in the respective frequency ranges is approximated to this target sound pressure ratio. Therefore, the individual difference in the inclination of the spectrum of the output signal of the detection unit (microphone) is accommodated by a simple control method.

The target sound pressure ratio setting unit may set the target sound pressure ratio so that the target sound pressure ratio is steadily increased as the inclination decreases in a predetermined noise range.

With this arrangement, an amplification amount is maintained within a permissible range because the target sound pressure ratio is variably set so as to be steadily increased as the inclination decreases in the noise range.

The noise range as used herein means a range of a value to be taken by the inclination acquired by the inclination acquiring unit when the noise actually occurs.

The inclination acquiring unit preferably acquires the inclination by determining, on the basis of the output signal of the detection unit, a ratio of sound pressures in at least two inclination reference frequency ranges which are less susceptible to the active noise cancellation than the resonance frequency range and the noise cancellation frequency range in which the noise is canceled by the sound output by the sound outputting unit.

With this unique arrangement, the inclination of the spectrum can be acquired relatively easily by determining the ratio of the sound pressures in the at least two inclination reference frequency ranges which are less susceptible to the active noise cancellation.

The adjustment unit preferably sets the gain at zero when no noise is present.

With this unique arrangement, the gain is not needlessly increased, because the gain is set at zero when no noise is present. Therefore, the active noise cancellation is not needlessly performed.

All the components of the active noise cancellation helmet are mounted in the helmet body, but this is not necessarily required. For example, the detection unit and the sound outputting unit may be mounted in the helmet body in association with the user's ear, and some of the other components may constitute a device separate from the helmet body.

A motor vehicle system according to a preferred embodiment of the present invention includes a vehicle body, and the aforementioned active noise cancellation helmet, wherein at least the detection unit and the sound outputting unit are mounted in the helmet body of the active noise cancellation helmet, and some of the components of the active noise cancellation helmet other than the detection unit and the sound outputting unit constitute a vehicle-side device provided in the vehicle body. The motor vehicle system further includes a communication unit that is arranged to allow for transmission of a signal between the vehicle-side device and the detection unit and between the vehicle-side device and the sound outputting unit.

With this unique arrangement, some of the components of the active noise cancellation helmet are disposed in the vehicle body.

A motor vehicle system according to another preferred embodiment of the present invention includes a vehicle body, the aforementioned active noise cancellation helmet, an audible information generating unit provided in the vehicle body and arranged to generate audible information, a transmission unit that is arranged to transmit the audible information generated by the audible information generating unit to the helmet body of the active noise cancellation helmet, and an audible information outputting unit provided in the helmet body and arranged to output the audible information transmitted by the transmission unit.

With this arrangement, the audible information from the audible information generating unit mounted in the vehicle body can be provided to the helmet wearer, while the noise in the helmet body is canceled irrespective of the individual differences between various users or wearers of the helmet. Thus, the helmet wearer can comfortably and reliably hear the provided audible information.

Examples of the audible information generating unit include a navigation system which provides audible guidance information, a mobile phone such as a cellular phone, a radio and an audio system.

Examples of the transmission unit include a wire communication unit that is arranged to connect the audible information generating unit to the helmet body via a cable, and a wireless communication unit for infrared communication or radio communication.

A typical example of the audible information outputting unit is a speaker provided in the helmet body. For example, a single speaker provided in the helmet body may be used as the audible information outputting unit and the sound outputting unit for the noise cancellation. Alternatively, separate speakers respectively defining the audible information outputting unit and the sound outputting unit for achieving the noise cancellation may be provided in the helmet body.

A method of canceling noise in a helmet according to a preferred embodiment of the present invention includes the steps of detecting noise in a helmet body by a detection unit, outputting a sound from a sound outputting unit for canceling the detected noise, processing an output signal of the detection unit through computation to generate a control signal, amplifying the generated control signal by an amplification unit and applying the amplified control signal to the sound outputting unit, acquiring a ratio of sound pressures in different frequency ranges on the basis of the output signal of the detection unit, and adjusting a gain of the amplification unit on the basis of the acquired sound pressure ratio so that a spectrum of the output signal of the detection unit is approximated to a predetermined target spectrum.

Thus, the active noise cancellation can accommodate the individual differences in auditory sound conduction function.

The method preferably further includes the step of acquiring an inclination of the spectrum of the output signal of the detection unit. In this case, the gain adjusting step preferably includes the step of adjusting the gain of the amplification unit on the basis of the acquired sound pressure ratio and the acquired inclination so that the spectrum of the output signal of the detection unit is approximated to the predetermined target spectrum.

Thus, the active noise cancellation can accommodate the individual differences in the spectrum of the output signal of the detection unit.

The foregoing and other elements, features, steps, characteristics and advantages of the present invention will become more apparent from the following detailed description of the preferred embodiments with reference to the attached drawings.

### BRIEF DESCRIPTION OF THE DRAWINGS

FIG. 1A is a block diagram illustrating the construction of an active noise cancellation helmet according to one preferred embodiment of the present invention;

FIG. 1B is an exterior view of the active noise cancellation helmet of FIG. 1A;

FIG. 2 is a diagram illustrating the construction of a control system of the active noise cancellation helmet according to the aforementioned preferred embodiment of the present invention;

FIG. 3 is a block diagram illustrating an exemplary digital circuit which performs active noise cancellation control according to the aforementioned preferred embodiment of the present invention;

FIG. 3A is a block diagram illustrating another exemplary digital circuit which performs active noise cancellation control according to the aforementioned preferred embodiment of the present invention;

FIG. 4 is a diagram for explaining the active noise cancellation control to be performed by the digital circuit of FIG. 3;

FIG. 5A is a diagram showing an effect that is achieved by the active noise cancellation control according to the aforementioned preferred embodiment of the present invention when great wind noise is present;

FIG. 5B is a diagram showing an effect that is achieved when small wind noise is present;

FIG. 5C is a diagram showing an effect that is achieved when no wind noise is present;

FIG. 6 is a block diagram illustrating further another exemplary digital circuit which performs active noise cancellation control according to the aforementioned preferred embodiment of the present invention;

FIG. 6A is a block diagram illustrating still another exemplary digital circuit which performs active noise cancellation control according to the aforementioned preferred embodiment of the present invention;

FIG. 7 is a diagram for explaining the active noise cancellation control to be performed by the digital circuit of FIG. 6;

FIGS. 8 and 8A are diagrams illustrating exemplary J_{d} functions (target sound pressure ratio function);

FIG. 9A is a diagram illustrating a spectrum having a steep inclination in a wind noise range;

FIG. 9B is a diagram illustrating a control method to be performed when the inclination is steep in the wind noise range;

FIG. 9C is a diagram illustrating an effect provided by the control method shown in FIG. 9B;

FIG. 10A is a diagram illustrating a spectrum having a gentle inclination in the wind noise range;

FIG. 10B is a diagram illustrating a control method to be performed when the inclination is gentle in the wind noise range;

FIG. 10C is a diagram illustrating an effect provided by the control method shown in FIG. 10B;

FIG. 11A is a diagram illustrating a flat spectrum having a zero inclination in a windless range;

FIG. 11B is a diagram illustrating a control method to be performed when the spectrum is flat in the windless range;

FIG. 11C is a diagram illustrating an effect provided by the control method shown in FIG. 11B;

FIG. 12A is a diagram illustrating a case where an inclination of a wind noise spectrum and a sound pressure at a resonance frequency are each expressed by a single parameter value;

FIG. 12B is a diagram illustrating a case where an inclination of a wind noise spectrum and a sound pressure at the resonance frequency are each expressed by an average of two parameter values;

FIG. 13 is a graph illustrating an individual difference in auditory sound conduction function;

FIG. 14 is a diagram illustrating an individual difference in the inclination of a wind noise spectrum;

FIG. 15 is a diagram illustrating the overall construction of a motor vehicle system including an active noise cancellation helmet according to another preferred embodiment of the present invention; and

FIG. 16 is a block diagram illustrating the electrical construction of the motor vehicle system of FIG. 15.

### DETAILED DESCRIPTION OF PREFERRED EMBODIMENTS

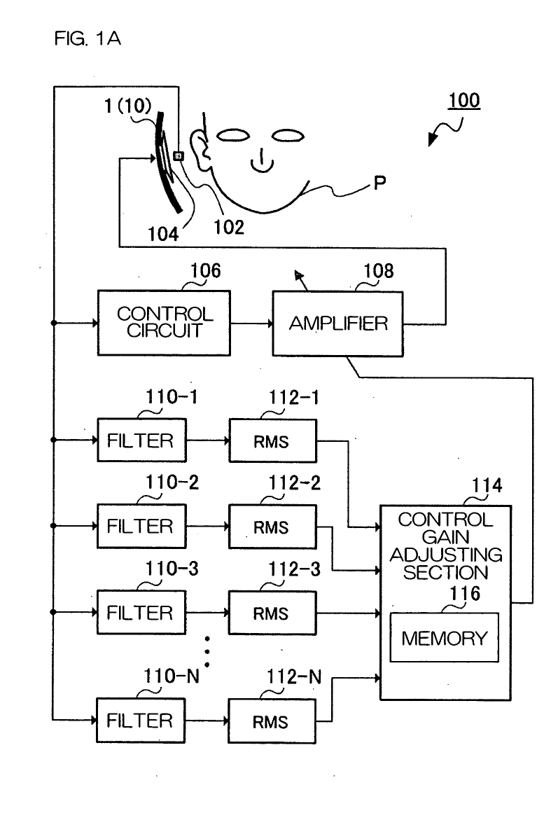

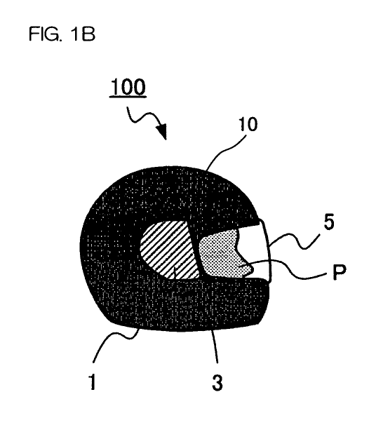

FIG. 1A is a block diagram illustrating the construction of an active noise cancellation helmet according to one preferred embodiment of the present invention, and FIG. 1B is an exterior view of the active noise cancellation helmet of FIG. 1A.

The active noise cancellation helmet 100 is an active noise cancellation device of a feedback type applied to a helmet. The active noise cancellation helmet 100 preferably includes a microphone (detection unit) 102 which detects noise (e.g., wind noise or other types of noise) in the helmet, a speaker (sound outputting unit) 104 which outputs a sound (secondary sound) for actively canceling the detected noise, a control circuit (signal generating unit) 106 which processes output signals of the microphone 102 through computation to generate a control signal for outputting the sound (secondary sound) for the noise cancellation, and an amplifier (amplification unit) 108 which amplifies the generated control signal and applies the amplified control signal to the speaker 104.

The microphone 102 and the speaker 104 are disposed at predetermined desired positions within a shell 1 of a helmet body 10. More specifically, as shown in FIG. 1A, the microphone 102 and the speaker 104 are located in a space that is adjacent to an ear of a user (helmet wearer) P when the user P wears the helmet body 10. Particularly, the microphone 102 is located in the vicinity of the user's ear between the user's ear and the speaker 104 so as to detect a sound that is close to a sound heard by the user P. The position of the microphone 102 is defined as a noise cancellation point. In FIG. 1B, a reference numeral 3 denotes a cover, and a reference numeral 5 denotes a shield.

The control circuit 106 samples an instantaneous value of a sound wave detected by the microphone 102 at the predetermined position (noise cancellation point) in the ear space within the helmet, and computes a control signal for driving the speaker 104 so that a sound pressure level at the noise cancellation point in the ear space is minimized. The control signal is applied to the speaker 104 via the amplifier 108, and the sound is output from the speaker 104 in the ear space on the basis of the control signal. Thus, the noise in the ear space adjacent to the user's ear is cancelled. That is, the control circuit 106 adaptively controls the output of the speaker 104 so as to minimize the sound at the position of the microphone 102.

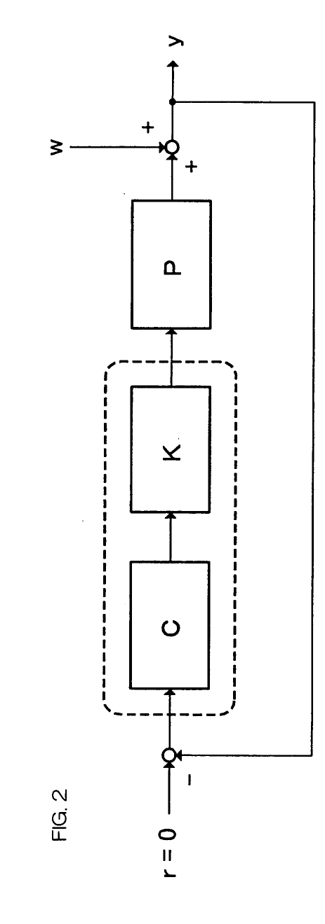

The basic principle of the feedback type active noise cancellation will be described with reference to FIG. 2. FIG. 2 is a diagram illustrating the construction of a control system of the active noise cancellation helmet according to this preferred embodiment. In FIG. 2, a reference character P denotes a frequency conduction function (auditory sound conduction function) to be controlled, a reference character C denotes a control filter (i.e., a frequency conduction function in the control circuit 106), and a reference character K denotes a control gain (the gain of the amplifier 108). A reference character y indicates the output of the microphone 102, and a reference character w indicates noise (e.g., wind noise). A reference character r indicates an input of the system, which is herein zero (0).

The sound heard by the user P is close to the output y of the microphone 102 and, therefore, the active noise cancellation helmet 100 aims at reducing the level of the output y of the microphone 102. In a known automatic control theory, the control filter C is designed in the form of an inverse of the auditory sound conduction function P, and the microphone output y is approximated to zero (0) by increasing the control gain K. However, it is difficult to design the control filter C in the form of the inverse of the auditory sound conduction function P in a full frequency range. If the control gain K is increased, the sound is progressively amplified to excess at a certain frequency (resonance frequency), resulting in divergence (howling). Thus, the noise cancellation and the excessive amplification are inextricably linked with each other. Therefore, the control gain K should be adjusted at a proper level in order to provide a sufficient noise cancellation effect while properly suppressing the amplification.

For example, an experiment reveals that, in a noise cancellation frequency range (noise cancellation range) of 100 Hz to 400 Hz, the active noise cancellation is effective, and the noise cancellation effect is increased as the control gain K is increased. On the other hand, the resonance frequency is about 2.5 kHz, at which the amplification effect is increased as the control gain K is increased. That is, when an attempt is made to reduce a control amount (here, the microphone output y) in a certain frequency range, the control amount is increased in another frequency range. This phenomenon is generally known as the “waterbed effect”.

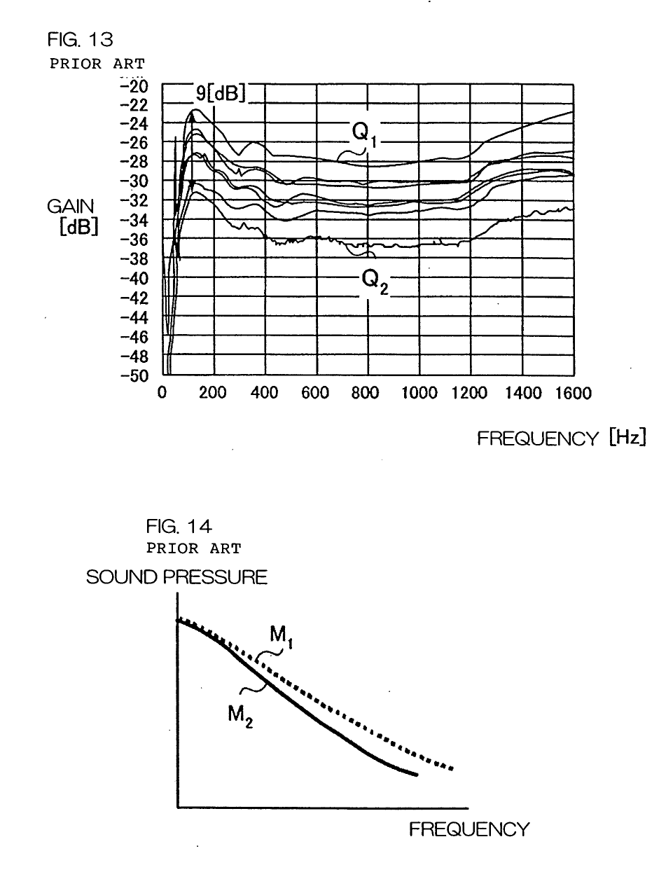

As previously mentioned, it is known that the auditory sound conduction function differs among individuals (see FIG. 13). That is, the phase of the auditory sound conduction function as well as the profile of the gain thereof (frequency dependency) do not depend much on individuals while the gain of the conduction function is entirely shifted depending on the users. If the control gain is evenly adjusted without consideration of the individual differences, as described above, the control gain K is excessively effective thereby resulting in divergence depending on the users or, conversely, is ineffective to reduce the noise cancellation effect to a level that is lower than expected without divergence. Therefore, if the gain to be controlled differs among individuals, it is necessary to adaptively adjust the control gain K.

For the adaptive adjustment of the gain (control gain K) of the amplifier 108 to accommodate the individual differences in this preferred embodiment, as shown in FIG. 1A, the active noise cancellation helmet 100 further includes a plurality of filters (N filters) 110-1 to 110-N which have different frequency characteristics to filter the output signals of the microphone 102, a plurality of effective value calculating sections (N effective value calculating sections) 112-1 to 112-N which calculate effective values (RMS values: Root Mean Square values) of output signals of the corresponding filters 110, and a control gain adjusting section (adjustment unit) 114 which adjusts the control gain K on the basis of the obtained plurality of effective values. An algorithm for the adjustment of the control gain K is stored in a memory 116 provided in the control gain adjusting section 114.

The N filters 110-1 to 110-N sample a necessary number of waveform segments (N waveform segments) in desired frequency ranges from the output signals of the microphone 102. Then, the effective values of the sampled waveform segments are calculated by the corresponding effective value calculating sections 112. The effective values correspond to sound pressures observed in the respective frequency ranges. Therefore, the effective value calculating sections 112 function as a sound pressure calculating unit.

The control gain adjusting section 114 also functions as a sound pressure ratio calculating unit which calculates a sound pressure ratio as a control index on the basis of the sound pressures (effective values) calculated for the respective frequency ranges, and adjusts the control gain K on the basis of the calculated sound pressure ratio so that the profile of the spectrum of the output signals of the microphone 102 is optimized. Specific methods for the adjustment of the control gain K will be described later.

The filters 110 are not limited to band-pass filters, but high-pass filters or low-pass filters may be used as the filters 110 when necessary. Alternatively, through-filters which pass the signals as they are may be used as the filters 110 when necessary.

In the calculation of the sound pressures in the respective frequency ranges, the effective values are not limited to the RMS values, but may be averages of absolute values of sound pressures. Alternatively, any effective values serving as an index of sound pressure levels in the unit of Pascal (Pa) may be used.

Although control methods according to this preferred embodiment can be implemented by either a digital circuit or an analog circuit, the digital circuit is preferably used for the control methods in the following explanation by way of example.

First Control Method

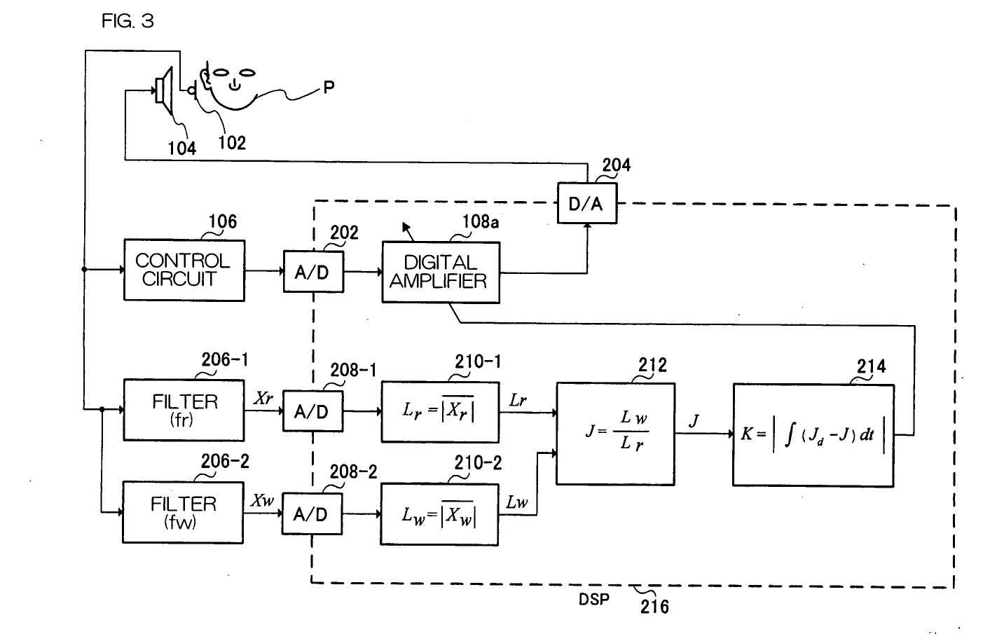

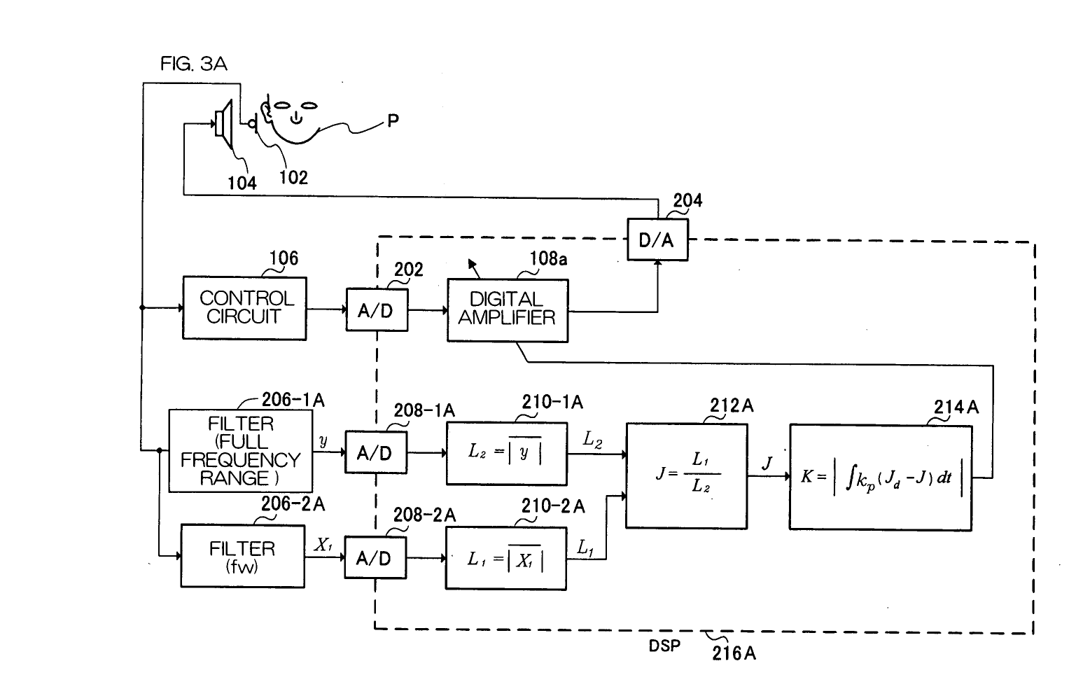

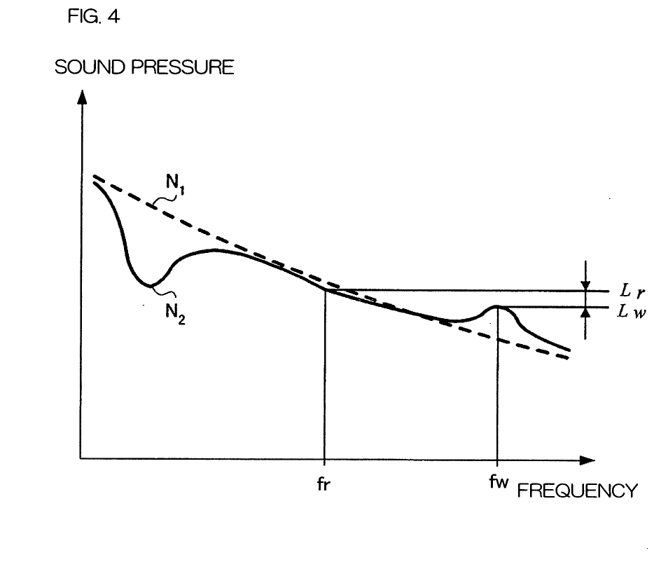

FIG. 3 is a block diagram illustrating an exemplary digital circuit which performs active noise cancellation control according to this preferred embodiment. FIG. 4 is a diagram for explaining the active noise cancellation control to be performed by the digital circuit of FIG. 3. In FIG. 3, elements corresponding to those shown in FIG. 1A will be denoted by the same reference characters as in FIG. 1A, and no repetitious explanation of these elements will be provided.

The output signals of the microphone 102 (sound pressure levels at the position of the microphone) are input to the control circuit 106. The control circuit 106 generates a control signal for driving the speaker 104 on the basis of the output signals of the microphone 102 according to a predetermined algorithm, and outputs the generated control signal to a digital amplifier 108a via an A/D converter 202. The digital amplifier 108a amplifies the control signal generated by the control circuit 106 with a control gain K, and outputs the amplified control signal to the speaker 104 via a D/A converter 204. The speaker 104 outputs a noise cancellation sound in the ear space on the basis of the input of the amplified control signal so as to cancel the noise.

On the other hand, the output signals of the microphone 102 (sound pressure levels at the position of the microphone) are also input to filters 206-1, 206-2. The filter 206-1 selectively passes signals in a predetermined frequency range (for example, having a center frequency fr), while the filter 206-2 selectively passes signals in another predetermined frequency range (for example, having a center frequency fw). The frequency range having the center frequency fr is less susceptible to active noise cancellation (ANC), and the center frequency fw is a resonance frequency (see FIG. 4). In FIG. 4, a reference character N₁ indicates a spectrum of the noise in the helmet before the ANC, and a reference character N₂ indicates a spectrum of noise in the helmet after the ANC.

The signals Xr, Xw passed through the filters 206-1, 206-2 are respectively input into sound pressure calculating sections 210-1, 210-2 via A/D converters 208-1, 208-2. The sound pressure calculating section 210-1 calculates an average (sound pressure) Lr of values of the signals Xr passed through the filter 206-1, and the sound pressure calculating section 210-2 calculates an average (sound pressure) Lw of values of the signals Xw passed through the filter 206-2 (see FIG. 4). The filter 206-2 and the sound pressure calculating section 210-2 function as a first acquisition unit which acquires a sound pressure in the resonance frequency range, while the filter 206-1 and the sound pressure calculating section 210-1 function as a second acquisition unit which acquires a reference sound pressure for comparison. The averages of the values of the signals passed through the respective filters may each be calculated, for example, as an RMS value or an average of absolute values of the signals.

The sound pressures Lr, Lw respectively calculated by the sound pressure calculating sections 210-1, 210-2 are input to a sound pressure ratio calculating section 212. The sound pressure ratio calculating section 212 calculates a ratio J (=Lw/Lr) of the sound pressures Lr, Lw.

The sound pressure ratio J calculated by the sound pressure ratio calculating section 212 is input to an adjustment section 214. The adjustment section 214 adjusts the control gain K (the gain of the digital amplifier 108a) on the basis of the input sound pressure ratio J through integration control (I control).

More specifically, a target value J_{d} (target sound pressure ratio) of the sound pressure ratio J is preliminarily determined from the following expression (1), and a deviation (J_{d}-J) of the sound pressure ratio J from the target value J_{d} is integrated with respect to time, and the absolute value of the integrated deviation is defined as the control gain K.
K=|∫(J_{d}−J)dt| (1)

That is, the sound pressures Lr, Lw in the predetermined frequency ranges fr, fw are determined through the filtering and the sound pressure calculation, and the control gain K is adjusted on the basis of the ratio J (=Lw/Lr) of the sound pressures Lr, Lw in the active noise cancellation control performed by this digital circuit.

In the circuit shown in FIG. 3, the digital amplifier 108a, the sound pressure calculating sections 210-1, 210-2, the sound pressure ratio calculating section 212 and the adjustment section 214 are preferably constituted, for example, by a digital signal processor (DSP) 216.

The frequency ranges for the sound pressures to be used for the calculation of the sound pressure ratio J (control index) are not limited to the frequency ranges fr, fw. For example, the control gain K may be adjusted by using the following expressions (2) to (5).

$$\begin{matrix}{L}_{1} ≡ \frac{1}{T} {\int }_{0}^{T} \vert {F}_{1} y ( t ) \vert ⅆ t & ( 2 ) \\ {L}_{2} ≡ \frac{1}{T} {\int }_{0}^{T} \vert y ( t ) \vert ⅆ t & ( 3 )\end{matrix}$$

J≡L₁/L₂ (4)
K=|∫kₚ(J_{d}−J)dt| (5)
wherein L₁ is an average of absolute values of the signals obtained by filtering the output signals y of the microphone 102 by a high-pass filter (having a center frequency fw) and corresponds to a sound pressure level in the resonance frequency range, and L₂ is an average of absolute values of the signals y obtained by passing the output signals y of the microphone 102 as they are and corresponds to a sound pressure level in a full frequency range as the reference frequency range. The ratio J (=L₁/L₂) of these absolute value averages indicates a proportion of a high frequency component (including a resonance frequency component) in the entire wind noise. In the expression (5), J_{d} is an optimum value (target value) of the sound pressure ratio J, and kₚ is a proper constant. Further, F₁ in the expression (2) indicates an operator corresponding to the high-pass filter mentioned above. That is, “F₁y(t)” is an expression of the result obtained by filtering the signal y(t) with the high-pass filter.

FIG. 3A is a block diagram illustrating another exemplary digital circuit preferably used for the adjustment of the control gain K using the expressions (2) to (5). In FIG. 3A, elements corresponding to those shown in FIG. 3 will be denoted by the same reference characters as in FIG. 3.

The output signals of the microphone 102 (sound pressure levels at the position of the microphone) are input to filters 206-1A, 206-2A. The filter 206-1A passes signals in a full frequency range, while the filter 206-2A corresponds to the operator F₁, and selectively passes signals in a frequency range (resonance frequency range) having the resonance frequency fw at the center thereof.

The signals y, X₁ passed through the filters 206-1A, 206-2A are respectively input into sound pressure calculating sections 210-1A, 210-2A via A/D converters 208-1A, 208-2A. The sound pressure calculating section 210-1A calculates an average (sound pressure) L₂ of values of the signals y passed through the filter 206-1A from the expression (3), and the sound pressure calculating section 210-2A calculates an average (sound pressure) L₁ of values of the signals X₁ passed through the filter 206-2A from the expression (2) (see FIG. 4). The filter 206-2A and the sound pressure calculating section 210-2A function as a first acquisition unit which acquires a sound pressure in the resonance frequency range, while the filter 206-1A and the sound pressure calculating section 210-1A function as a second acquisition unit which acquires a sound pressure in the reference frequency range. The averages of the values of the signals passed through the respective filters may each be calculated, for example, as an RMS value or an average of absolute values of the signals.

The sound pressures L₁, L₂ respectively calculated by the sound pressure calculating sections 210-1A, 210-2A are input to a sound pressure ratio calculating section 212A. The sound pressure ratio calculating section 212A calculates a ratio J (=L₁/L₂) of the sound pressures L₁, L₂ from the expression (4).

The sound pressure ratio J calculated by the sound pressure ratio calculating section 212A is input into an adjustment section 214A. The adjustment section 214A adjusts the control gain K (the gain of the digital amplifier 108a) on the basis of the input sound pressure ratio J through integration control (I control) based on the expression (5).

The expressions (1), (5) for determining the control gain each have the following two functions. A first function is to adjust the control gain K so that the sound pressure ratio J is approximated to the target value J_{d}. A second function is to allow the control gain K to have a value that is not less than zero (0). The first function is provided by the integration control (I control), while the second function is provided by the absolute value calculation in the expressions (1), (5). The integration control eliminates a steady-state deviation of the sound pressure ratio J from the target value J_{d} which can be eliminated by neither proportional control (P control) nor differential control (D control) Therefore, the control method preferably includes at least the integration control, but may also include the proportional control and/or the differential control in combination with the integration control.

The absolute value calculation prevents a malfunction (divergence) which may otherwise occur when the control gain K adjusted by the digital circuit has a negative value.

More specifically, the gain K is calculated by integrating the deviation (J_{d}-J) with respect to time. If the sound pressure ratio J is smaller than the target value J_{d}, the gain K is gradually increased and, at the same time, the sound pressure ratio J is increased. Conversely, if the sound pressure ratio J is greater than the target value J_{d}, the gain K is gradually reduced and, at the same time, the sound pressure ratio J is reduced. Thus, the sound pressure ratio J converges on the target value J_{d}, whereby the spectrum of the output signals of the microphone 102 is optimized.

On the other hand, if the control gain K was reduced to a negative value, divergence (howling) would occur. In this preferred embodiment, however, the control gain K is calculated as the absolute value of the integrated value for prevention of the divergence. Therefore, the control gain K has a lower limit of 0.

Since it is known that the sound pressure ratio J is steadily increased with the control gain K, the control gain K can be adjusted at an optimum level through the integration control based on the expression (1) or (5).

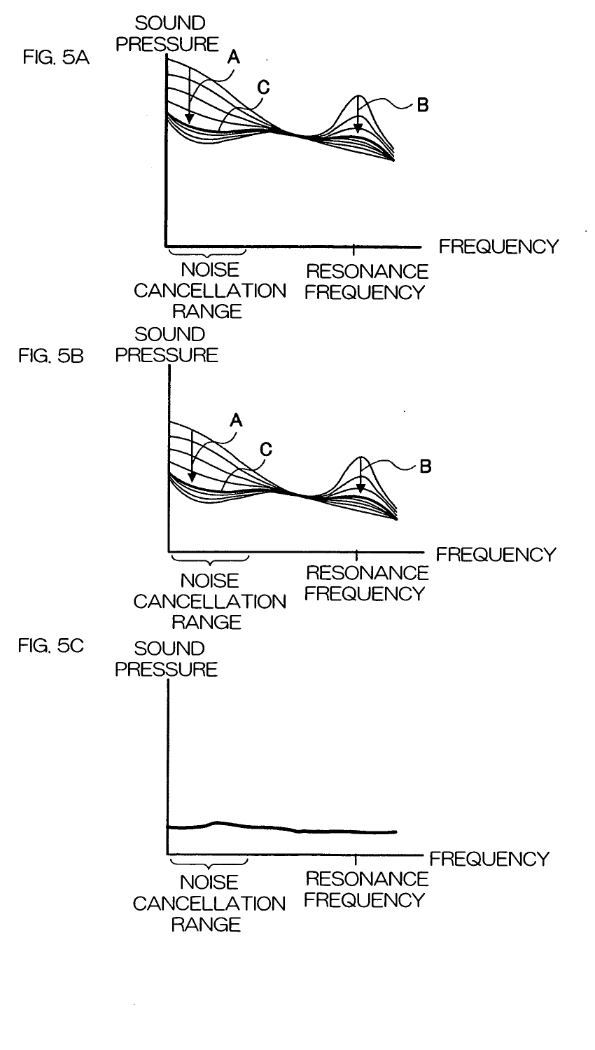

FIGS. 5A, 5B and 5C are diagrams for explaining effects achieved by the active noise cancellation control according to this preferred embodiment. Particularly, FIG. 5A is a diagram showing an effect achieved when great wind noise is present, and FIG. 5B is a diagram showing an effect achieved when small wind noise is present. FIG. 5C is a diagram showing an effect achieved when no wind noise is present.

The active noise cancellation control according to this preferred embodiment, e.g., the active noise cancellation control based on the expressions (2) to (5), eliminates the individual difference in the auditory sound conduction function, and is optimized irrespective of the level of the wind noise.

That is, the active noise cancellation control according to this preferred embodiment aims at approximating the profile of the noise (wind noise) spectrum to a target spectrum profile. An exemplary target spectrum profile is such that the sound pressure L₂ is ten times as great as the sound pressure L₁ (with a sound pressure difference of +20 dB), i.e., the target value J_{d} in the expression (5) is set at J_{d}=1/10. Then, the control gain K is adjusted through the calculation of the expression (5) so that the ratio J (=L₁/L₂) of the current sound pressures L₁, L₂ is equalized with the target value J_{d}. That is, the control is not dependent upon the absolute values of the microphone output signals, because the ratio of the sound pressures in the different frequency ranges is used.

Further, when the sound pressure L₁ in the resonance frequency range is amplified through the active noise cancellation (ANC) control, the user P recognizes the level of the amplified sound pressure L₁ (loudness) by comparison with the level of the sound pressure L₁ observed before the ANC. In other words, where a sound pressure in a frequency range f₃ that is less susceptible to the ANC is defined as L₃, the user P recognizes the loudness by comparing the level of the sound pressure L₃ observed after the ANC with the level of the sound pressure L₁ observed after the ANC. This is because the level of the sound pressure L₃ is rarely changed by the ANC (though influenced by the whole noise level). Therefore, a proper relationship (noise pressure ratio after ANC) which ensures moderate cancellation of the noise in the noise cancellation range (in a major wind noise frequency range to be subjected to the ANC) while suppressing the loudness of the noise in the resonance frequency range can be determined between the sound pressures L₃ and L₁. Such a proper relationship is not limited to that determined between the sound pressures L₃ and L₁ in the predetermined frequency ranges, but can be determined between sound pressures in every possible combination of frequencies. In general, an optimum spectrum profile can be determined which ensures hearing comfort after the ANC.

Since the sound pressure L₂ indicating the sound pressure level in the full frequency range is not changed by the ANC, the sound pressure ratio J=L₁/L₂ indicates the spectrum profile dependent upon the control gain K. Therefore, the optimum spectrum profile can be provided by adjusting the control gain K to approximate the sound pressure ratio J to the target value J_{d}.

In FIGS. 5A and 5B, for example, the control gain K is increased if the noise level is high in a low frequency range (noise cancellation range) or the sound pressure ratio J is low. Thus, the noise level in the low frequency range is reduced as indicated by an arrow A in FIGS. 5A and 5B. On the other hand, if the noise level is high in a high frequency range (resonance frequency range) or the sound pressure ratio J is high, the control gain K is reduced. Thus, the noise level in the high frequency range is reduced as indicated by an arrow B in FIGS. 5A and 5B. The control gain K is thus automatically controlled through the integration control based on the expression (1) or (5), whereby the spectrum profile is approximated to the optimum target spectrum profile.

In addition, as shown in FIGS. 5A and 5B, the target spectrum profile is not dependent upon the entire noise level. That is, the profile of the target spectrum is not varied by the level of the wind noise, so that the target value J_{d} realizing the target spectrum can be set at a constant level. Therefore, the optimum control can be performed irrespective of the level of the wind noise by adjusting the control gain K through the integration control using the sound pressure ratio J.

The final goal of the active cancellation of the wind noise is to approximate the wind noise spectrum profile to the optimum spectrum profile to ensure the hearing comfort. Although a spectrum profile for every user P can be approximated to the target spectrum profile by adjusting the control gain K, the value of the control gain K for the approximation differs from user to user due to the individual difference in the auditory sound conduction function. For elimination of the individual differences, therefore, the spectrum profile should be directly monitored when the control gain K is adjusted to approximate the spectrum profile to the optimum spectrum profile. This is also realized by the integration control using the sound pressure ratio J.

If the wind noise is not present, the control gain K is set at zero (0), and the active noise cancellation is not performed as shown in FIG. 5C. Therefore, there is no possibility that the noise signal is needlessly amplified. That is, background noise (mainly a high frequency noise component) is dominant in the microphone output signals without the wind noise. Therefore, the proportion of the high frequency noise component in the entire noise is increased as compared with a case where the wind noise is present. Accordingly, the value of the sound pressure ratio J (=L₁/L₂ or Lw/Lr) exceeds the target value J_{d}, and the control gain K is continuously reduced, for example, according to the expression (5). However, the control gain K never has a negative value because of the absolute value calculation. Therefore, the control gain K finally converges on K=0, so that the output of the speaker 104 is reduced to zero (0). That is, the active noise cancellation is not performed.

Second Control Method

Although the first control method is directed to the elimination of the individual differences in the auditory sound conduction function, the inclination of the wind noise spectrum also differs among individuals as described above. It is known that the wind noise spectrum is typically such that the sound pressure is reduced as a frequency increases, but the inclination of the spectrum differs among individuals (see FIG. 14). The sound pressure ratio J is dependent upon the inclination of the spectrum. Therefore, if the target value J_{d} is set at a constant level, the individual difference in the inclination of the wind noise spectrum cannot reliably be eliminated.

FIG. 14 is a diagram showing the individual differences in the inclination of the wind noise spectrum. As shown in FIG. 14, the sound pressure of the wind noise is generally increased as the frequency decreases, and is generally reduced as the frequency increases. However, the inclination of the spectrum differs among individuals. In FIG. 14, the inclination of the spectrum for a user M₁ is less steep than the inclination of the spectrum for a user M₂. If the inclination is more gentle than usual, the high frequency noise component occupies a greater proportion of the entire wind noise. Even if the wind noise is not sufficiently cancelled by the ANC control (i.e., if the amplification in the resonance frequency range is insufficient), the sound pressure ratio J has a relatively great value. Therefore, the control gain K is adjusted at a lower level than usual, so that the noise cancellation effect is reduced. Conversely, if the inclination is steeper than usual, the sound pressure ratio J has a relatively small value. Therefore, the control gain K is adjusted at a higher level than usual, so that the amplification in the resonance frequency range is excessive.

In view of this, a method for the active noise cancellation control will be described, which can accommodate not only the individual differences in the auditory sound conduction function but also the individual differences in the inclination of the wind noise spectrum.

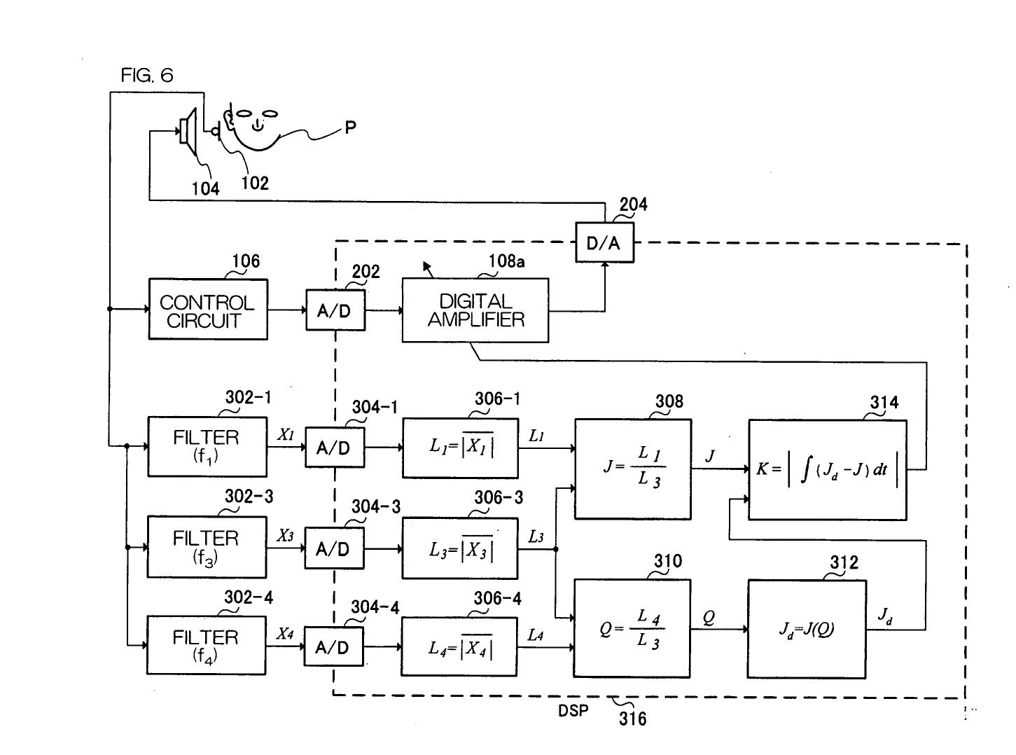

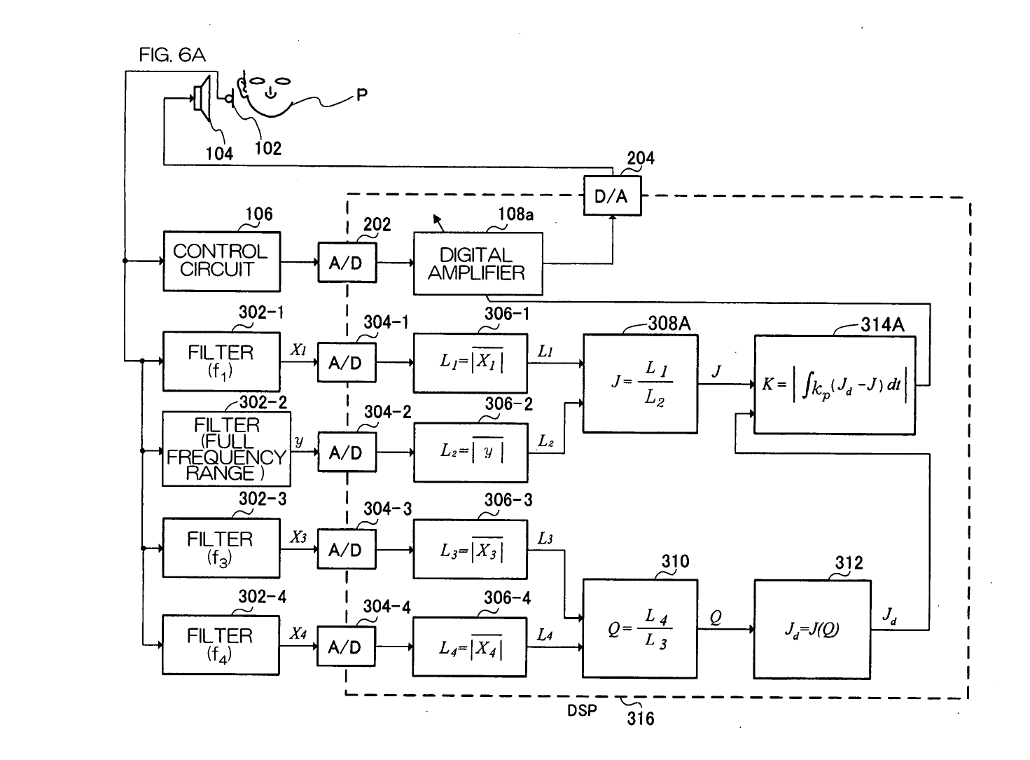

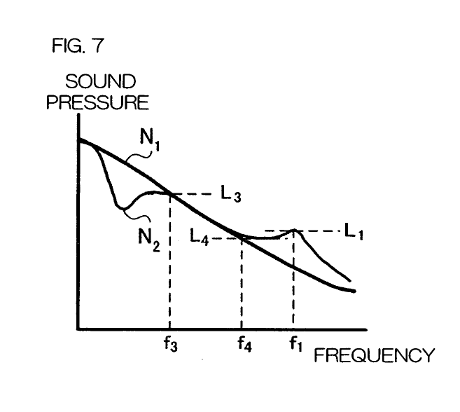

FIG. 6 is a block diagram illustrating further another exemplary digital circuit which performs active noise cancellation control according to the present preferred embodiment. FIG. 7 is a diagram for explaining the active noise cancellation control to be performed by this digital circuit. In FIG. 6, elements corresponding to those shown in FIG. 3 will be denoted by the same reference characters as in FIG. 3, and no repetitious explanation of these common elements will be provided.

In contrast to the first control method described with reference to FIG. 3 or FIG. 3A in which the target value J_{d} of the sound pressure ratio J is constant, this control method has a feature that the target value J_{d} is variably set as a function of the wind noise spectrum inclination.

In this control method, the output signals of the microphone 102 (sound pressure levels at the position of the microphone) are input to three filters 302-1, 302-3, 302-4. The filter 302-1 selectively passes signals in a predetermined frequency range (for example, having a frequency f₁). The filter 302-3 selectively passes signals in another predetermined frequency range (for example, having a frequency f₃), and the filter 302-4 selectively passes signals in further another predetermined frequency range (for example, having a frequency f₄). The frequency f₁ is the resonance frequency, and the frequencies f₃, f₄ are in inclination reference frequency ranges which are used for determination of the inclination of a spectrum and are less susceptible to the active noise cancellation (ANC) control (see FIG. 7). In FIG. 7, a reference character N₁ indicates a spectrum of noise in the helmet before the ANC, and a reference character N₂ indicates noise in the helmet after the ANC.

The signals X₁, X₃, X₄ passed through the filters 302-1, 302-3, 302-4 are respectively input into sound pressure calculating sections 306-1, 306-3, 306-4 via A/D converters 304-1, 304-3, 304-4. The sound pressure calculating section 306-1 calculates an average (sound pressure) L₁ of values of the signals X₁ passed through the filter 302-1. The sound pressure calculating section 306-3 calculates an average (sound pressure) L₃ of values of the signals X₃ passed through the filter 302-3, and the sound pressure calculating section 306-4 calculates an average (sound pressure) L₄ of values of the signals X₄ passed through the filter 302-4 (see FIG. 7). The averages of the values of the signals passed through the respective filters may each be calculated, for example, as an RMS value or an average of absolute values of the signals.

The sound pressures L₁, L₃ respectively calculated by the sound pressure calculating sections 306-1, 306-3 are input into a sound pressure ratio calculating section 308. The sound pressure ratio calculating section 308 calculates a ratio J (=L₁/L₃) of the input sound pressures L₁, L₃.

On the other hand, the sound pressures L₃, L₄ respectively calculated by the sound pressure calculating sections 306-3, 306-4 are input into a sound pressure ratio calculating section 310 that functions as an inclination acquiring unit which acquires the inclination of the microphone output signal spectrum. The sound pressure ratio calculating section 310 calculates a ratio Q (=L₄/L₃) of the input sound pressures L₃, L₄. The sound pressure ratio Q indicates the inclination of the microphone output signal spectrum, i.e., the inclination of the wind noise spectrum. In general, the ratio Q has a value that is not greater than 1 when the wind noise is dominant, and has a value that is close to 1 when the background noise is dominant without the wind noise.

The sound pressure ratio Q calculated by the sound pressure ratio calculating section 310 is input to a target value calculating section (target sound pressure ratio setting unit) 312. The target value calculating section 312 calculates a target value J_{d} on the basis of the input sound pressure ratio Q from a predetermined J_{d} function (target sound pressure ratio function). The J_{d} function is a function of the sound pressure ratio Q (i.e., the wind noise spectrum inclination) for the target value J_{d} of the sound pressure ratio J as will be described later.

Then, the sound pressure ratio J calculated by the sound pressure ratio calculating section 308 and the target value J_{d} calculated by the target value calculating section 312 are input to an adjustment section 314. The adjustment section 314 adjusts the control gain K (the gain of the digital amplifier 108a) on the basis of the input sound pressure ratio J and the input target value J_{d} through integration control (I control).

More specifically, a deviation (J_{d}-J) of the sound pressure ratio J from the target value J_{d} is integrated with respect to time through the following expression (6), and the control gain K is calculated as the absolute value of the deviation.
K=|∫(J_{d}−J)dt| (6)

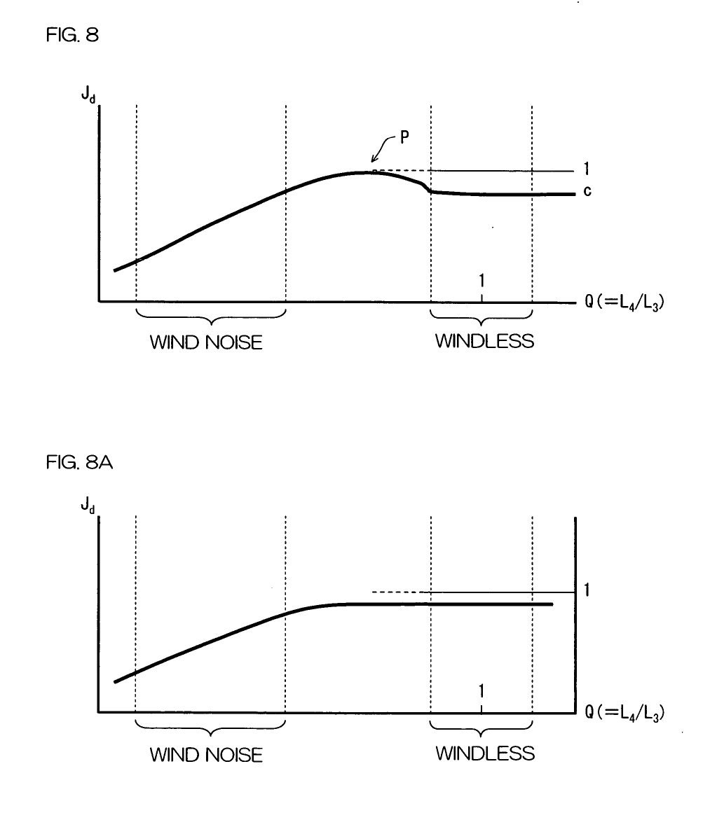

FIG. 8 is a diagram illustrating an example of the J_{d} function. As shown in FIG. 8, the J_{d} function has different characteristics in a range of the ratio Q (wind noise range or noise range) in which the wind noise is present and in a range of the ratio Q (windless range or noise less range) in which the wind noise is not present. More specifically, the target value J_{d} is preferably steadily increased with the ratio Q in the wind noise range in which the ratio Q (=L₄/L₃) is smaller. In the windless range in which the ratio Q is close to 1, the target value J_{d} preferably has a value that is smaller than 1. In FIG. 8, a peak p of the target value J_{d} is present between the wind noise range and the windless range. The target value J_{d} is steadily reduced from this peak p with the ratio Q in the windless range, and is kept at a constant value C smaller than 1 in the windless range. The constant value C is smaller than a target value J_{d} at the peak p and greater than a lower limit of the target value J_{d} in the windless range.

FIG. 8A shows another example of the J_{d} function. In this example, the target value J_{d} is steadily increased with respect to Q in the wind noise range, and is substantially kept at a constant not more than 1 in the windless range. There is no peak between the wind noise range and the windless range.

In FIGS. 8 and 8A, the upper limit of the target value J_{d} is generally equal to 1. In some cases, however, it is reasonable that the upper limit of the target value J_{d} is set at a value that is greater than 1 or at a value that is smaller than 1. In the windless range shown in FIGS. 8 and 8A, the target value J_{d} is set at the constant value irrespective of the ratio Q, but may be steadily reduced with the ratio Q.

More specifically, if the ratio Q (=L₄/L₃) is smaller in the wind noise range, i.e., if the inclination of the spectrum is steep, the target value J_{d} of the sound pressure ratio J (=L₁/L₃) is reduced to set the sound pressure L₁ at a relatively low level. Conversely, if the ratio Q is greater in the wind noise range, i.e., if the inclination of the spectrum is gentle, the target value J_{d} of the sound pressure ratio J is increased to set the sound pressure L₁ at a relatively high level. Thus, the J_{d} function is defined such that the target value J_{d} is increased as the ratio Q increases in the wind noise range.

On the other hand, the inclination of the spectrum is further reduced to be generally flat in the windless range (Q≈1). Therefore, the target value J_{d} is set at a value not greater than 1. Thus, the control gain K is reduced to reduce the sound pressure L₁. The control gain K is finally reduced to zero (0), thereby obviating the need for the ANC.

In the circuit shown in FIG. 6, the digital amplifier 108a, the sound pressure calculating sections 306-1, 306-3, 306-4, the sound pressure ratio calculating sections 308, 310, the target value calculating section 312 and the adjustment section 314 are constituted, for example, by a digital signal processor (DSP) 316.

The frequency ranges for the sound pressures to be used for the calculation of the sound pressure ratio J and the sound pressure ratio Q (spectrum inclination) are not limited to the frequency ranges f₁, f₃, f₄. The control gain K may be adjusted by using the following expressions (7) to (13).

$$\begin{matrix}{L}_{1} ≡ \frac{1}{T} {\int }_{0}^{T} \vert {F}_{1} y ( t ) \vert ⅆ t & ( 7 ) \\ {L}_{2} ≡ \frac{1}{T} {\int }_{0}^{T} \vert y ( t ) \vert ⅆ t & ( 8 ) \\ {L}_{3} ≡ \frac{1}{T} {\int }_{0}^{T} \vert {F}_{3} y ( t ) \vert ⅆ t & ( 9 ) \\ {L}_{4} ≡ \frac{1}{T} {\int }_{0}^{T} \vert {F}_{4} y ( t ) \vert ⅆ t & ( 10 )\end{matrix}$$

J≡L₁/L₂ (11)
J≡J_{d}(L₄/L₃) (12)
K=|∫kₚ(J_{d}−J)dt| (13)

The expressions (7), (9), (11), (13) are identical to the expressions (2), (3), (4), (5), respectively. That is, the sound pressure ratio J (=L₁/L₂) indicates the proportion of the resonance frequency component in the wind noise. Further, F₁, F₃, F₄ indicate filter operators respectively corresponding to filters with center frequencies f₁, f₃, and f₄, respectively. The results obtained by filtering the signal y(t) with those filters are indicated as “F₁y(t)”, “F₃y(t)”, “F₄y(t)”, respectively.

Through this control, the control gain K can be adjusted so as to accommodate the individual differences in the inclination of the wind noise spectrum without needlessly performing the active noise cancellation (ANC) in the windless state.

FIG. 6A is a block diagram illustrating still another exemplary digital circuit for the adjustment of the control gain K using the expressions (7) to (13). In FIG. 6A, components corresponding to those shown in FIG. 6 will be denoted by the same reference characters as in FIG. 6.

The output signals of the microphone 102 are input to a through-filter 302-2 which passes signals in the full frequency range as well as the three filters 302-1, 302-3, 302-4 (corresponding to operators F₁, F₃, F₄, respectively). Signals y passed through the filter 302-2 are converted into digital signals by an A/D converter 304-2, and then input into a sound pressure calculating section 306-2. The sound pressure calculating section 306-2 calculates an average (sound pressure) L₂ of values of the signals y passed through the filter 302-2 (an average sound pressure level in the full frequency range) (see the expression (8)). The average of the values of the signals passed through the respective filters may be calculated, for example, as an RMS value or an average of absolute values of the sound pressures.

The sound pressure L₁ calculated by the sound pressure calculating sections 306-1 (see the expression (7)) and the sound pressure L₂ calculated by the sound pressure calculating section 306-2 are input into a sound pressure ratio calculating section 308A. The sound pressure ratio calculating section 308A calculates a ratio J (=L₁/L₂) of the input sound pressures L₁, L₂ (see the expression (11)).

On the other hand, the sound pressure L₃ calculated by the sound pressure calculating section 306-3 (see the expression (9)) and the sound pressure L₄ calculated by the sound pressure calculating section 306-4 (see the expression (10)) are input into the sound pressure ratio calculating section 310 as in the case shown in FIG. 6.

Then, the sound pressure ratio J calculated by the sound pressure ratio calculating section 308A and the target value J_{d} calculated by the target value calculating section 312 (see the expression (12)) are input into an adjustment section 314A. The adjustment section 314A adjusts the control gain K (the gain of the digital amplifier 108a) on the basis of the input sound pressure ratio J and the input target value J_{d} through integration control (I control)(see the expression (13)).

The expressions (6), (13) which define the control gain each have two functions as in the first control method. A first function is to adjust the control gain K so that the sound pressure ratio J is approximated to the target value J_{d}. A second function is to allow the control gain K to have a value not smaller than zero (0). That is, the gain K is determined by integrating the deviation (J_{d}-J) with respect to time. Thus, if the sound pressure ratio J is smaller than the target value J_{d}, the gain K is gradually increased and, at the same time, the sound pressure ratio J is increased. Conversely, if the sound pressure ratio J is greater than the target value J_{d}, the gain K is gradually reduced and, at the same time, the sound pressure ratio J is reduced. Thus, the sound pressure ratio J converges on the target value J_{d}, whereby the output signal spectrum of microphone 102 is optimized. On the other hand, if the control gain K was reduced to a negative value, divergence (howling) would occur. In this preferred embodiment, however, the control gain K is calculated as the absolute value of the integrated value for prevention of the divergence.

Effects and advantages achieved by the control method when the inclination of the spectrum is steep in the wind noise range, when the inclination of the spectrum is gentle in the wind noise range and when the spectrum is flat in the windless range will hereinafter be described.

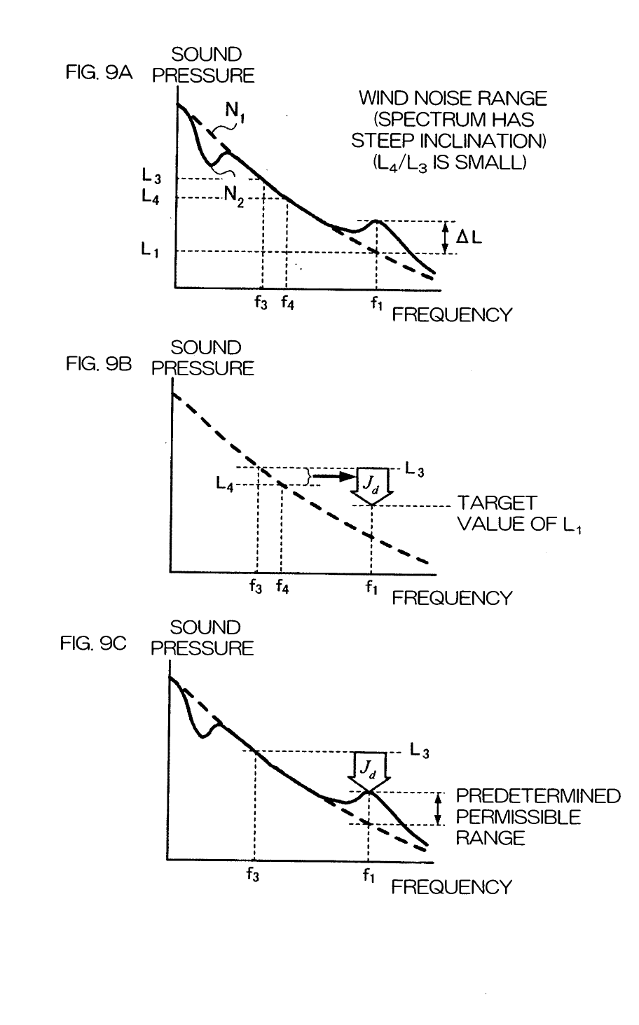

FIG. 9A illustrates an exemplary spectrum having a steep inclination in the wind noise range, and FIG. 9B illustrates a control method to be performed in this case. FIG. 9C illustrates an effect of this control method.

If the inclination of the spectrum is steep in the wind noise range, i.e., if the ratio Q (=L₄/L₃) is smaller (see FIG. 9A) , the control gain K is controlled so as to maintain an amplification amount ΔL within a predetermined permissible range in the resonance frequency range f₁. More specifically, the ratio Q (=L₄/L₃) is smaller so that the target value J_{d} is set at a smaller value according to the J_{d} function shown in FIG. 8 or 8A. Thus, the target value of the sound pressure L₁ is reduced relative to the sound pressure L₃, so that the length of a white arrow shown in FIG. 9B is increased. Therefore, the control gain K is adjusted so as to reduce the sound pressure ratio J (=L₁/L₃) (see FIG. 9B). As a result, the amplification amount ΔL is maintained within the predetermined permissible range (see FIG. 9C).

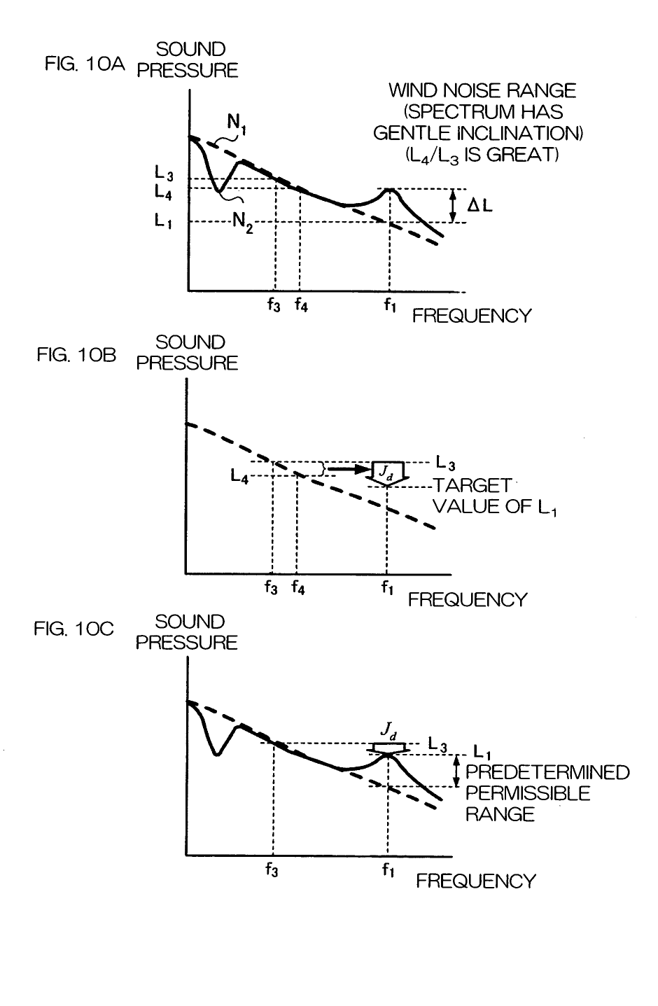

FIG. 10A illustrates an exemplary spectrum having a gentle inclination in the wind noise range, and FIG. 10B illustrates a control method to be performed in this case. FIG. 10C illustrates an effect of this control method.

If the inclination of the spectrum is gentle in the wind noise range, i.e., if the ratio Q (=L₄/L₃) is greater (see FIG. 10A), the control gain K is controlled so as to maintain the amplification amount ΔL within the predetermined permissible range in the resonance frequency range f₁. More specifically, the ratio Q (=L₄/L₃) is greater so that the target value J_{d} is set at a greater value according to the J_{d} function shown in FIG. 8 or 8A. Thus, the target value of the sound pressure L₁ is increased relative to the sound pressure L₃, so that the length of a white arrow shown in FIG. 10B is reduced. Therefore, the control gain K is adjusted so as to increase the sound pressure ratio J (=L₁/L₃) (see FIG. 10B). As a result, the amplification amount ΔL is maintained within the predetermined permissible range (see FIG. 10C).

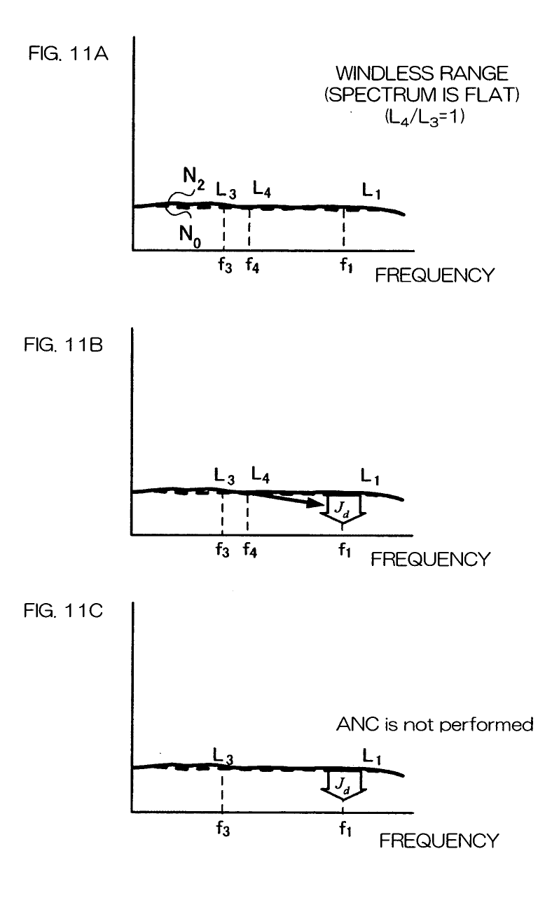

FIG. 11A illustrates a flat spectrum observed in the windless range, and FIG. 11B illustrates a control method to be performed in this case. FIG. 11C illustrates an effect of this control method.

If the spectrum is flat in the windless range (see FIG. 11A), the active noise cancellation (ANC) is not performed. More specifically, the target value J_{d} is set at a value that is much smaller than 1 according to the J_{d} function shown in FIG. 8 or 8A. At this time, the sound pressure L₁ is nearly equal to the sound pressure L₃, so that the value J is nearly equal to 1. Further, the control gain K is adjusted so as to approximate the value J to the target value J_{d} for reduction of the sound pressure L₁. More specifically, the control gain K is progressively reduced. However, the absolute value is calculated in the expression (13), so that the control gain K takes a value not smaller than zero (0). Therefore, the control gain K is set at zero (0) (see FIG. 11B). As a result, the output of the speaker 104 is nullified, so that the active noise cancellation (ANC) is not performed. In FIG. 11A, a reference character N₀ denotes background noise.

In this control method, the target value J_{d} of the sound pressure ratio J is changed according to the inclination Q of the wind noise spectrum, so that the individual difference in the inclination of the wind noise spectrum can be accommodated.

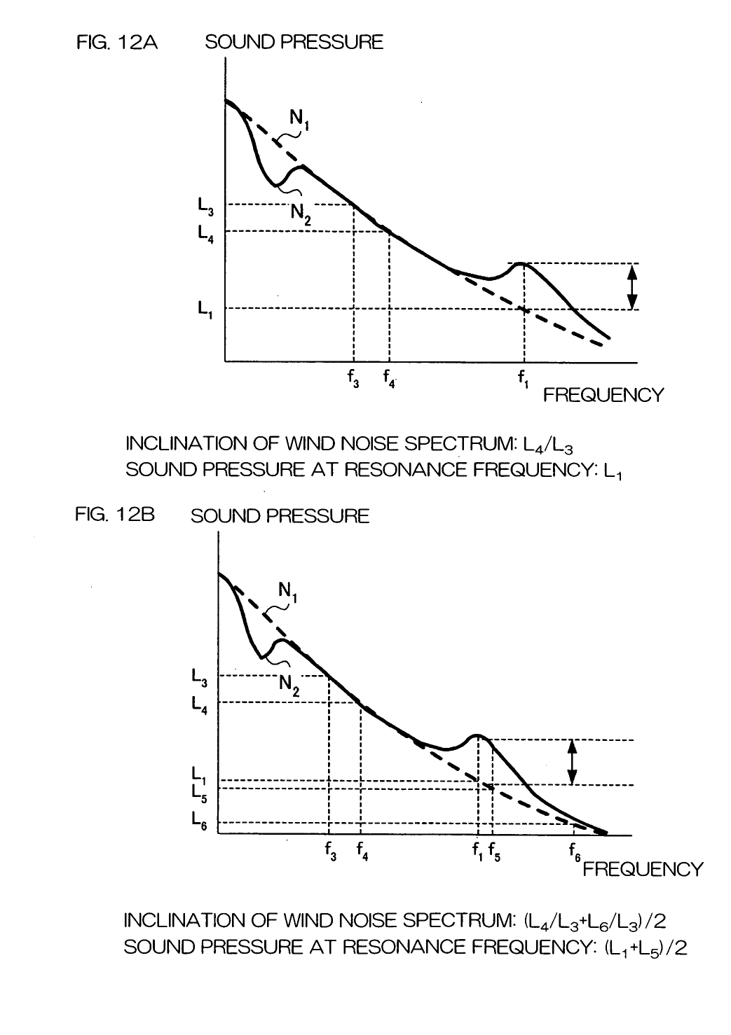

In the aforementioned control method, the parameters each preferably have a single parameter value, but may each have a plurality of parameter values. For example, the sound pressure ratio J may include, for example, a plurality of sound pressure ratios (M sound pressure ratios) J₁ to J_{M}. More specifically, the sound pressure ratio J is calculated as an average of the plurality of sound pressure ratios J₁ to J_{M}. Thus, the accuracy is improved. For example, the inclination of the wind noise spectrum and the sound pressure at the resonance frequency each have a single parameter value in FIG. 12A. On the other hand, the inclination of the wind noise spectrum and the sound pressure at the resonance frequency are each represented by an average of two parameter values in FIG. 12B.

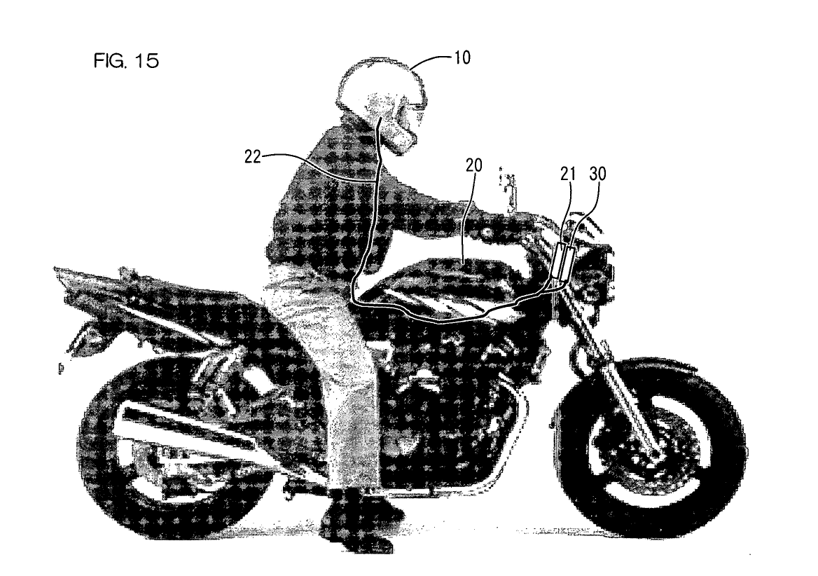

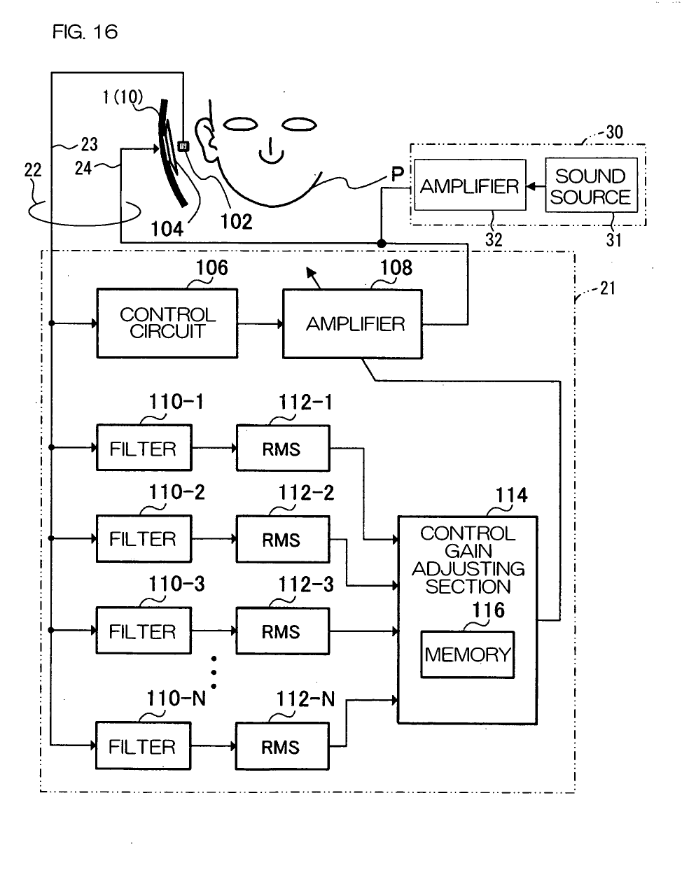

FIG. 15 is a diagram illustrating the overall construction of a motor vehicle system including the aforementioned active noise cancellation helmet according to another preferred embodiment of the present invention. FIG. 16 is a block diagram illustrating the electrical construction of the motor vehicle system. In FIGS. 15 and 16, elements corresponding to those shown in FIGS. 1A and 1B will be denoted by the same reference characters as in FIGS. 1A and 1B.

In this preferred embodiment, only the microphone 102 and the speaker 104 (e.g., a panel speaker) out of the components of the active noise cancellation helmet are mounted in the helmet body 10, and the other elements including the control circuit 106 are provided in an ANC controller amplifier 21 as a vehicle-side device mounted in a vehicle body 20 of a two-wheeled vehicle as an exemplary motor vehicle. The ANC controller amplifier 21 is connected to the microphone 102 and the speaker 104 via a wire harness 22 including a plurality of cables bundled together.

The wire harness 22 is a communication unit which includes a microphone signal line 23 for inputting the output signals of the microphone 102 into the ANC controller amplifier 21 and a sound signal line 24 for applying the noise cancellation control signal to the speaker 104 from the ANC controller amplifier 21.

An audible information generating device 30 is provided in the vehicle body 20, and connected to the sound signal line 24. The audible information generating device 30 includes a sound source 31 which generates a sound signal, and an amplifier 32 which amplifies the sound signal generated by the sound source 31 and outputs the amplified sound signal to the sound signal line 24. Therefore, the sound signal line 24 also functions as transmission unit which transmits the sound signal to the helmet body 10.

The speaker 104 provided in the helmet body 10 constantly outputs the noise cancellation sound on the basis of the control signal, and outputs a sound on the basis of the sound signal generated by the audible information generating device 30 when necessary. That is, the speaker 104 also functions as audible information outputting unit which outputs audible information. Thus, the wearer of the helmet body 10 hears the audible information output by the audible information generating device 30 with the wind noise being properly cancelled.

The audible information generating device 30 may be a navigation device which provides an audible guidance message, an audio device such as a radio or an audio player, or a mobile phone (for example, having a mail reading-out function as well as a basic conversation function).

The ANC controller amplifier 21 and the audible information generating device 30 are not necessarily required to be connected to the helmet body 10 via the cables, but signal transmission may be achieved by wireless communication such as infrared communication.

The ANC controller amplifier 21 may have an internal construction selected from those shown in FIGS. 3, 3A, 6 and 6A.

This preferred embodiment is also applicable to a four-wheeled vehicle, as long as a driver of the vehicle is required to wear a helmet.

While the present invention has been described in detail by way of the preferred embodiments thereof, it should be understood that the foregoing disclosure is merely illustrative of the technical principles of the present invention but not limitative of the same. The spirit and scope of the present invention are to be limited only by the appended claims.

This application corresponds to Japanese Patent Application No. 2003-403745 filed in the Japanese Patent Office on Dec. 2, 2003, the disclosure of which is incorporated herein by reference.

1. An active noise cancellation helmet comprising:
a detection unit that is arranged to detect noise in a helmet body;
a sound outputting unit that is arranged to output a sound for canceling the noise detected by the detection unit;
a signal generating unit that is arranged to process an output signal of the detection unit through computation to generate a control signal;
an amplification unit that is arranged to amplify the control signal generated by the signal generating unit and to apply the amplified control signal to the sound outputting unit;
a sound pressure ratio acquiring unit that is arranged to acquire a ratio of sound pressures in different frequency ranges on the basis of the output signal of the detection unit; and
an adjustment unit that is arranged to adjust a gain of the amplification unit on the basis of the sound pressure ratio acquired by the sound pressure ratio acquiring unit so as to approximate a spectrum of the output signal of the detection unit to a predetermined target spectrum.

2. An active noise cancellation helmet as set forth in claim 1, wherein the detection unit is provided within the helmet body so as to be located in the vicinity of a user's ear when a user wears the helmet body.

3. An active noise cancellation helmet as set forth in claim 1, wherein the detection unit includes at least one microphone.

4. An active noise cancellation helmet as set forth in claim 1, wherein the sound outputting unit includes at least one speaker.

5. An active noise cancellation helmet as set forth in claim 1, wherein the signal generating unit includes a control circuit.

6. An active noise cancellation helmet as set forth in claim 1, wherein the amplification unit includes at least one amplifier.

7. An active noise cancellation helmet as set forth in claim 1, wherein the sound pressure ratio acquiring unit includes:
a plurality of filters having different frequency characteristics and arranged to filter the output signal of the detection unit;
a sound pressure calculating unit that is arranged to process output signals of the respective filters to calculate the sound pressures in the respective frequency ranges; and
a sound pressure ratio calculating unit that is arranged to calculate the sound pressure ratio as a control index on the basis of the sound pressures calculated for the respective frequency ranges by the sound pressure calculating unit.

8. An active noise cancellation helmet as set forth in claim 1, wherein the sound pressure ratio acquiring unit includes:
a first acquisition unit that is arranged to acquire a sound pressure in a resonance frequency range on the basis of the output signal of the detection unit;
a second acquisition unit that is arranged to acquire a reference sound pressure as a reference for comparison on the basis of the output signal of the detection unit; and
a sound pressure ratio calculating unit that is arranged to calculate a ratio of the sound pressure acquired for the resonance frequency range by the first acquisition unit to the reference sound pressure acquired by the second acquisition unit for the comparison.

9. An active noise cancellation helmet as set forth in claim 8, wherein the reference sound pressure to be acquired by the second acquisition unit is a sound pressure in a reference frequency range which is less susceptible to active noise cancellation than the resonance frequency range and a noise cancellation frequency range in which the noise is canceled by the sound output by the sound outputting unit.

10. An active noise cancellation helmet as set forth in claim 9, wherein the reference frequency range is a full frequency range.

11. An active noise cancellation helmet as set forth in claim 1, wherein the adjustment unit is arranged to adjust the gain of the amplification unit so that the sound pressure ratio acquired by the sound pressure ratio acquiring unit is approximated to a target sound pressure ratio corresponding to the predetermined target spectrum.

12. An active noise cancellation helmet as set forth in claim 1, further comprising an inclination acquiring unit that is arranged to acquire an inclination of the spectrum of the output signal of the detection unit, wherein the adjustment unit is arranged to adjust the gain of the amplification unit on the basis of the sound pressure ratio acquired by the sound pressure ratio acquiring unit and the inclination acquired by the inclination acquiring unit so that the spectrum of the output signal of the detection unit is approximated to the predetermined target spectrum.

13. An active noise cancellation helmet as set forth in claim 12, wherein the adjustment unit includes a target sound pressure ratio setting unit that is arranged to variably set a target sound pressure ratio for the predetermined target spectrum according to the inclination acquired by the inclination acquiring unit, and the adjustment unit is arranged to adjust the gain so that the sound pressure ratio acquired by the sound pressure ratio acquiring unit is approximated to the target sound pressure ratio set by the target sound pressure ratio setting unit.

14. An active noise cancellation helmet as set forth in claim 13, wherein the target sound pressure ratio setting unit is arranged to set the target sound pressure ratio so that the target sound pressure ratio is steadily increased as the inclination decreases in a predetermined noise range.

15. An active noise cancellation helmet as set forth in claim 12, wherein the inclination acquiring unit is arranged to acquire the inclination by determining, on the basis of the output signal of the detection unit, a ratio of sound pressures in at least two inclination reference frequency ranges which are less susceptible to active noise cancellation than the resonance frequency range and a noise cancellation frequency range in which the noise is canceled by the sound output by the sound outputting unit.

16. An active noise cancellation helmet as set forth in claim 1, wherein the adjustment unit is arranged to set the gain of the amplification unit at zero when no noise is present.

17. A motor vehicle system comprising:
a vehicle body; and
an active noise cancellation helmet including a detection unit that is arranged to detect noise in a helmet body, a sound outputting unit that is arranged to output a sound for canceling the noise detected by the detection unit, a signal generating unit that is arranged to process an output signal of the detection unit through computation to generate a control signal, an amplification unit that is arranged to amplify the control signal generated by the signal generating unit and applies the amplified control signal to the sound outputting unit, a sound pressure ratio acquiring unit that is arranged to acquire a ratio of sound pressures in different frequency ranges on the basis of the output signal of the detection unit, and an adjustment unit that is arranged to adjust a gain of the amplification unit on the basis of the sound pressure ratio acquired by the sound pressure ratio acquiring unit so as to approximate a spectrum of the output signal of the detection unit to a predetermined target spectrum; wherein
at least the detection unit and the sound outputting unit are mounted in the helmet body of the active noise cancellation helmet; and
some of the components of the active noise cancellation helmet other than the detection unit and the sound outputting unit constitute a vehicle-side device provided in the vehicle body;
the motor vehicle system further comprising a communication unit that is arranged to transmit a signal between the vehicle-side device and the detection unit and between the vehicle-side device and the sound outputting unit.

18. A motor vehicle system comprising:
a vehicle body;
an active noise cancellation helmet;
an audible information generating unit provided in the vehicle body and arranged to generate audible information;
a transmission unit that is arranged to transmit the audible information generated by the audible information generating unit to a helmet body of the active noise cancellation helmet; and
an audible information outputting unit provided in the helmet body and arranged to output the audible information transmitted by the transmission unit,
the active noise cancellation helmet including:
a detection unit that is arranged to detect noise in the helmet body;
a sound outputting unit that is arranged to output a sound for canceling the noise detected by the detection unit;
a signal generating unit that is arranged to process an output signal of the detection unit through computation to generate a control signal;
an amplification unit that is arranged to amplify the control signal generated by the signal generating unit and applies the amplified control signal to the sound outputting unit;
a sound pressure ratio acquiring unit that is arranged to acquire a ratio of sound pressures in different frequency ranges on the basis of the output signal of the detection unit; and
an adjustment unit that is arranged to adjust a gain of the amplification unit on the basis of the sound pressure ratio acquired by the sound pressure ratio acquiring unit so as to approximate a spectrum of the output signal of the detection unit to a predetermined target spectrum.

19. A motor vehicle system as set forth in claim 18, wherein the audible information generating unit includes at least one of a navigation system which provides audible guidance information, a mobile phone, a radio and an audio system.

20. A motor vehicle system as set forth in claim 18, wherein the transmission unit includes at least one of a wire communication unit that is arranged to connect the audible information generating unit to the helmet body, and a wireless communication unit for infrared communication or radio communication.

21. A motor vehicle system as set forth in claim 18, wherein the audible information outputting unit includes at least one speaker provided in the helmet body.

22. A method of canceling noise in a helmet comprising the steps of:
detecting noise in a helmet body by a detection unit;
outputting a sound from a sound outputting unit for canceling the detected noise;
processing an output signal of the detection unit through computation to generate a control signal;
amplifying the generated control signal by an amplification unit and applying the amplified control signal to the sound outputting unit;
acquiring a ratio of sound pressures in different frequency ranges on the basis of the output signal of the detection unit; and
adjusting a gain of the amplification unit on the basis of the acquired sound pressure ratio so that a spectrum of the output signal of the detection unit is approximated to a predetermined target spectrum.

23. A method as set forth in claim 22, further comprising the step of acquiring an inclination of the spectrum of the output signal of the detection unit, wherein the gain adjusting step includes the step of adjusting the gain of the amplification unit on the basis of the acquired sound pressure ratio and the acquired inclination so that the spectrum of the output signal of the detection unit is approximated to the predetermined target spectrum.
